(19) United States
(12) Patent Application Publication
(10) Pub. No.: US 2008/0206773 A1
(43) Pub. Date: Aug. 28, 2008

(54) DNA Fingerprinting Using Allelic Specific Oligonucleotide Reversed DOT BLOT (ASO-RDB) Flow Through Hybridization Process and Device

(76) Inventor: Joseph Wing On Tam, Hong Kong (HK)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/044,126

(22) Filed: Mar. 7, 2008

Related U.S. Application Data
(63) Continuation of application No. 10/293,248, filed on Nov. 9, 2002, now abandoned.

(51) Int. Cl. C12Q 1/68; C12M 1/34; C07H 21/04

(52) U.S. Cl. 435/6; 435/287.2; 536/24.3

(57) ABSTRACT

The present invention disclosed the use of single nucleotide polymorphism (SNP) as the detection assay for human identification. Using the reversed dot-blot format and the flow through hybridization process, the process can be more efficient, less expensive and with similar or better power of exclusion in definitive identification. The present method can be applied to any other organisms.

### CROSS REFERENCE

This application is a continuation app'l of U.S. Ser. No. 10/293,248, filed Nov. 9, 2002, which claims priority of U.S. Ser. No. 60/346,133, filed Nov. 7, 2001, the contents of which are incorporated in their entireties by reference into this application.

### BACKGROUND OF THE PRESENT INVENTION

1. Field of Invention

The present invention relates to method of making definitive identification of a human or any organism by DNA analysis and the device thereof.

2. Description of Related Arts

DNA fingerprinting by RFLP first introduced in 1985 (Gill P, Jeffreys A J, Werrett D J) for human identification was subsequently applied to other organisms. In human it was widely accepted as the best forensic tool for identification of suspects in criminal cases, paternity disputes and often used as the distinct human ID code. Recently the relatively time consuming RFLP method is mostly replaced by the high throughput automation processes. Using PCR amplification of analyzing the number of short tandem repeat (STR), first discovered in 1991 (Edwards A, Civitello A, Hammond H A, Caskey C T), from 10, 16 or 18 (or more) loci in the human genome, single cell identification is possible. However, both STR and/or VNTR are not only still relatively expensive because the methods require the use of sophisticated equipment or labor intensive time consuming process like the Southern Blotting Hybridization but also sporadic mutations (Chakraborty R, Stivers D N.) may reduce the power for definitive identification. Furthermore, our STR data (Tam J W et.al. to be published) suggested that the frequency of mutation, in cancer patients in particular, is not uncommon. Single nucleotide polymorphism (SNP) should have as high, if not more, discriminating power as VNTR or STR systems for forensic or individual personal identification. Hence new alternative method is needed. The present invention presents such an alternative with supporting data.

### SUMMARY OF THE PRESENT INVENTION

Now that the human genome and many other organisms have been sequenced and mapped. Although within any species the general DNA sequencing information is very similar but each and every one has each own distinct sets of information. Hence many scientists try to characterize disease-related variation among populations. Anthropologists use genetic variation to reconstruct our species' history, and to understand the role of culture and geography in the global distribution of human variation. Single nucleotide polymorphism (SNP) data can service these purpose (Weiss K M 1998). Indeed SNP can be used for genotyping (Brightwell G, Wycherley R, Waghorn A. 2002). Hence with the use of allele specific oligonucleotide (ASO)-arrays, the number of SNP to provide adequate discriminating power is easily attainable. We use our membrane-based semi-array ASO-RDB Flow-Through hybridization format to achieve such goal (see U.S. Pat. No. 5,741,647 for details). In principle we could use the SNP of sufficient number anywhere in the genome for discriminating purpose. However, this may compromises the accuracy of paternity and kinship analyses because of the variability of mutation rate in different part of the genome. Hence highly polymorphism sites or points in the genome where the mutation rate is relatively low (e.g. in the coding region, but not limit to, meaning any regions that satisfy the said conditions of relatively low mutation rate) to ensure the inherence nature for kinship identification. Our preliminary data using SNPs from 8 highly polymorphic chromosome loci, comprising 25 SNPs suffice to get enough of discrimination power for forensic exclusion and human identification. In construction of the polymorphic frequency database, on each site we have sequenced DNA samples from 50-150 unrelated individuals. The kinship analyses of 20 families were performed in parallel with the STR Profiler plus human identity kit and the results were 100% in agreement. Although more data may be needed for forensic validation to achieve higher discrimination power enough for global application, this SNP-based Flow Through format has proven to be a good alternative for human identification. In addition to data already accumulated and analyzed, expansion of the Data Base accumulation is in progress.

### BRIEF DESCRIPTION OF THE DRAWINGS

FIG. 1 is the diagram of the method for obtaining the SNP data base for the invention.

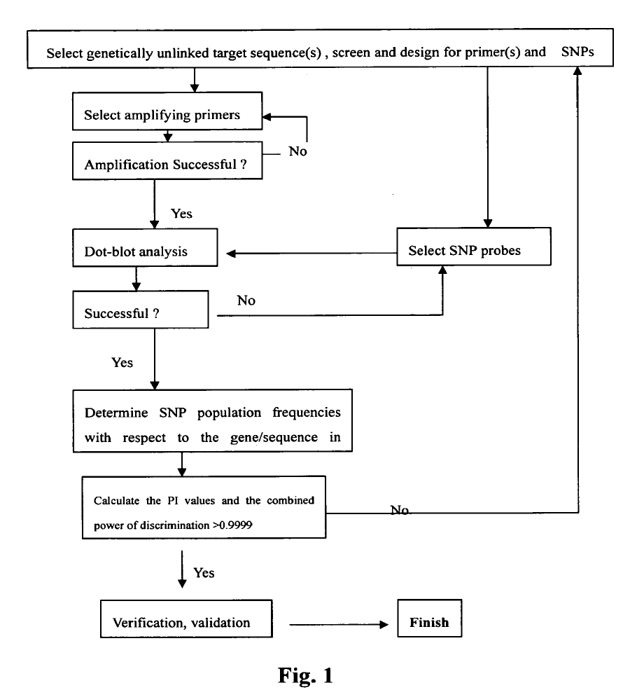

FIG. 2 is one of the example image data for identification of two individuals.

### DETAILED DESCRIPTION OF THE INVENTION

The following is the general procedure for the present invention:
(a) Select SNP sites and determine the power of exclusion
   1 Select the appropriate SNP oligonucleotide probes for the capture of the specific target sequences to be analyzed by either screening data from the GENBANK™ or perform population screening by sequencing the target genes or target DNA segments to get the SNP profile and population frequencies.
   2 From these data determine the SNP sites to be used for fingerprinting based on the polymorphic frequency and to evaluate if the sites are indeed not hot spots for mutation within a population by sequencing the random sampling.
   3 Then determine the number of SNP and calculate the total heterozygosity to determine the exclusion power.
(b) Perform SNP profile detection
   1 Then design the appropriate primers for amplification and the SNP-probes for hybridization detection.
   2 Amplify target sequences and perform the SNP profile analyses by Flow-through Hybridization process using the device depicted in U.S. Pat. No. 6,020,187.
   3 Then compare with known sequence data for accuracy evaluation.
   4 Modify the probes and testing conditions for accuracy. The RDB SNP data are verified by DNA sequencing.
(c) Validation
   Proceed for validation with random samples.

### EXAMPLE

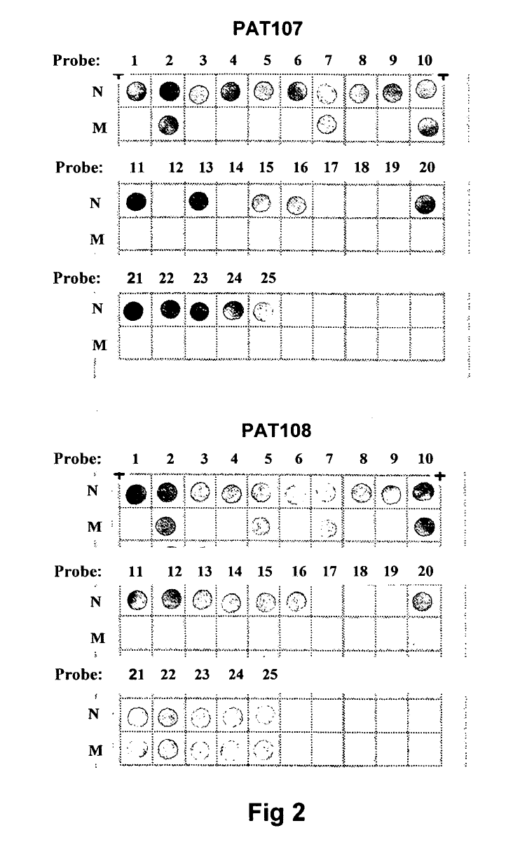

We have sequenced eight gene clusters and 55 segment sequenced with 50 to 400 individual samples for determining the SNP sites. FIG. 2 showed one of the panels we used for such fingerprinting each of which has been compared with the STR Profiler Plus fingerprinting kit from Applied Biosystems Inc for identification. Table I showed the loci used for such determination in the Figure I. Other probes and primers for other candidate genes/sequences are being tested. Genes partially tested include Globin genes for Thalassemia, BRCAs, ApoE, Collagens, p53, G6PD deficiency alleles and HLA DP, DQ and DR. In principle, any known SNPs of any organisms with adequate data to perform genetic analysis can be tested or detected by the Flow-through Hybridization Method.

### REFERENCE

1 Chakraborty R, Stivers D N. Paternity exclusion by DNA markers: effects of paternal mutations. J Forensic Sci 1996 July; 41(4): 671-7
2 Edwards A, Civitello A, Hammond H A, Caskey C T. DNA typing and genetic mapping with trimeric and tetrameric tandem repeats. Am J Hum Genet. 1991 October; 49(4): 746-56. Gill P, Jeffreys A J, Werrett D J. Forensic application of DNA ‘fingerprints’. Nature. 1985 Dec. 12-18; 318(6046): 577-9.
3 Tam Joseph Wing On, Flow Through Nucleic Acid Hybridisation Device, U.S. Pat. No. 6,020,187.
4 Weiss K M. In search of human variation. Genome Res 1998 July; 8(7): 691-7 Zhao L P, Aragaki C, Hsu L, Quiaoit F. Mapping of complex traits by single-nucleotide polymorphisms. Am J Hum Genet 1998 July; 63(1): 225-40

1. A flow-through hybridization method for nucleic acid assay for determination of the identification of an organism comprising:
a. the flow-through hybridization device;
b. a matrix material capable of immobilizing the single nucleotide polymorphism (SNP) oligonucleotide capture probe for capturing the target DNA for analysis.

2. The hybridization device, according to claim 1, whereas the device can be any kind comprising a hybridization chamber, a matrix and the device for liquid flow through process directing through the matrix for capturing the target molecules.

3. The matrix according to claim 1, whereas the matrix can be any material capable of the immobilizing SNP probes for capturing target DNA.

4. The SNP probe according to claim 1, can be any oligonucleotide sequence determined from the genome of the organism having polymorphism for said population.

5. The SNP probe according the claim 1, whereas the number of SNP oligonucleotides can be any number in combination as long as it achieves the exclusion power for definitive identification.
